(19) United States
(12) Patent Application Publication
(10) Pub. No.: US 2003/0064101 A1
(43) Pub. Date: Apr. 3, 2003

(54) Floating osmotic device for controlled release drug delivery

(75) Inventors: Bharat Pravinchandra Mehta, Mumbai (IN); Madhukant Mansukhlal Doshi, Mumbai (IN); Milind Dattatraya Joshi, Thane (IN)

Correspondence Address: LACKENBACH SIEGEL, ONE CHASE ROAD, SCARSDALE, NY, 10583, US

(73) Assignee: J.B. Chemicals & Pharmaceuticals Limited, Mumbai (IN)

(21) Appl. No.: 09/992,897

(22) Filed: Nov. 6, 2001

(30) Foreign Application Priority Data
Sep. 25, 2001 (IN) 922/MUM/2001

(51) Int. Cl. A61K 9/24

(52) U.S. Cl. 424/473

(57) ABSTRACT

The present invention provides a novel floating osmotic device that is capable of delivering a first active agent in an outer coat immediately followed by continuous controlled delivery of second active agent from the osmotic core while the dosage form floats in the fluid of the environment. The floating osmotic device has of a compressed core containing active agent, a semipermeable membrane surrounding the core that is permeable to surrounding fluid and impermeable to the active agent, and an outer coating of a gas generating ingredient, a gelling agent, and a second active agent. Particular embodiments of the invention provide floating osmotic devices in which all two active agents are similar or dissimilar.

### BACKGROUND OF THE INVENTION

1. Field of the Invention

This invention generally relates to pharmaceutical compositions. This invention relates in particular to such compositions in the form of floating osmotic devices for controlled delivery of one or more active agents.

This invention relates more particularly to floating osmotic devices for immediate delivery of a first active agent followed by continuous controlled delivery of a second agent, which may be same or different from the first active agent, while the device or dosage form floats in the fluid of the environment (e.g., the stomach), thereby being retained in the environment for an extended period of time.

2. Description of the Prior Art

Oral ingestion is the most preferred route of administration for various types of active agents, thereby providing a convenient method of effectively achieving both local and systemic action. However, the absorption of drugs may not be uniform over the entire gastrointestinal tract. Some drugs (e.g., methyidopa and captopril) are absorbed only from the stomach to the upper part of the intestine. Hence, there is a clear need to hold such drugs in the stomach for an extended period of time so as to achieve maximum and uniform absorption. In the case of drugs intended for the treatment of gastric disorders (e.g., ofloxacin in the treatment of H. pylori infection), it would be beneficial to achieve close adherence of the drugs to gastric mucosa. Hence these drugs also need to be retained in the stomach for optimum efficacy.

An important factor affecting the absorption of orally administered drug through gastrointestinal tract is transit time in gastrointestinal tract. Some drugs are required for local action in stomach as in case of ulcers, some drugs are absorbed only from the upper part of gastrointestinal tract. For example, ciprofloxacin is absorbed only from the stomach to the jejunum. The sparingly soluble drugs are administered several times per day since the solubility of drug decreases the time required for drug dissolution becomes less adequate and the gastrointestinal transit time becomes significant factor that interferes with drug absorption. These problems can be overcome with the floating osmotic device of the present invention.

The use of bioadhesive polymers for gastric retention is reviewed in the literature (see Longer et al., J. Pharm. Sci., 74, 406, 1985; Gurney and Junginger (Eds.), Bioadhesion—Possibilities and Future Trends, Wiss. Verlagsgesellschaft, Stuttgart (1990)). An oral controlled release formulation comprising calcium alginate and sodium bicarbonate has been described, which was found to increase the bioavailibility of Riboflavin (Ingani et al., Int. J. Pharm., 35, 157-164, 1987). Tablets and pellets with increased gastric retention and bioadhesion are described in PCT Publication No. WO94/00112 where natural gums, plant extracts, sucralfates, acrylic acid or methacrylic acid derivatives are employed to prolong the retention of drugs in the stomach for the treatment of gastric disorders. However, the use of bioadhesive systems to modify gastrointestinal transit has been abandoned since these mucoadhesive polymers are not able to control or slow down significantly the transit of solid delivery systems such as tablets/pellets (see Verma, Drug Dev. Ind. Pharm., 26(7), 695, 2000).

Low density (i.e., floating) systems in the form of pellets and tablets have also been reported (Mazer et al., J. Pharm. Sci, 77, 647,1988). But these systems do not provide an extended period of residence in the stomach unless administered along with a meal. U.S. Pat. No. 4,106,120 describes floating minicapsules containing sodium bicarbonate and coated with hydrophilic polymers. Similar floating granules are described by U.S. Pat. No. 4,884,905, whereas U.S. Pat. No. 5,198,229 describes floating capsules. U.S. Pat. No. 6,207,197 describes gastro-retentive controlled release compositions comprising microspheres containing a drug in an inner core, a rate controlling membrane of a hydrophilic polymer, and an outer layer of a bioadhesive cationic polymer. It is known that multiparticulate systems such as granules, microspheres, and microcapsules get distributed over the length of the gastrointestinal tract.

All the above systems describe gastro-retentive compositions which are either intended for immediate release, or for controlled delivery of drugs in matrix and/or reservoir type systems, which pose problems of bioavailibility fluctuations due to gastric pH variations. Moreover, the release of drugs from matrix and/or reservoir type controlled release systems is affected by hydrodynamic conditions of the body.

Another important factor affecting the absorption of orally administered drug through gastrointestinal tract is the length of time that the drug is present in the gastrointestinal tract. Better control over in-vivo performance of an active agent is possible if delivery is from an osmotic core following zero order kinetics. (Theeuwes et al., Br. J. Clin. Pharmacol., 19, 69S-76S, 1985). In addition, a drug released from an osmotic drug delivery system can exhibit significant in-vitro/in-vivo correlation. (Gupta et al., Eur. J. Pharm. Biopharm., 42, 74-81, 1996; Chao et al., J. Pharm. Sci., 82, 432-435, 1991). Representative literature on osmotic pump delivery devices include U.S. Pat. Nos. 3,797,492; 4,008,719; 4,265,874; 4,439,195; 4,610,686; 4,662,880; 4,986,987; 5,147,654; 5,200,194; and 5,869,096. Osmotic delivery has been proved to be advantageous for delivering many drugs in controlled manner. Attempts are also been made to modify the osmotic pumps to achieve efficient drug release as per the need. Cussler et al. (U.S. Pat. No. 5,827,538) described an osmotic device which on imbibement of water vapor provides controlled release of drug. These devices minimize the incompatibilities between active agents and ions or other dissolved materials in aqueous media. Additionally the high water fluxes attendant with vapor permeable membrane facilitated delivery of drug with low solubility. U.S. Pat. No. 5,869,096 describes osmotic device for delivering drug with hydrogel driving member consisting of a layer of hydrophilic polymer which operates to diminish the volume occupied by the active agent thereby delivering the agent from the device at a controlled rate over extended period of time. Gastrointestinal Therapeutic Systems (GITS) of nifedipine (Swanson et al., Am. J. Med., 83(Suppl 6B), 3-9, 1987), and glipizide (U.S. Pat. No. 5,545,413 to Kuczynski, et al.) were reported to be significantly more effective than the corresponding immediate-release formulations. Known osmotic delivery systems include those described in U.S. Pat. Nos. 6,132,420; 5,827,538; 6,270,787; and 5,795,591.

### SUMMARY OF THE INVENTION

It is an object of this invention to provide a novel pharmaceutical composition in the form of floating osmotic device that allows immediate delivery of active agent present in an external core which in turn causes the system to float and facilitates the delivery of the drug in second osmotic core continuously in controlled manner.

It is another object of the present invention to provide a drug delivery device that exhibits immediate release of one active ingredient present in an outer coat followed by controlled release of second active agent incorporated in a osmotic core.

It is also an object of the invention to provide a drug delivery device that floats and thereby is retained in the stomach

It is a further object of the present invention to provide such a floating drug delivery device that immediately releases a first drug from an outer coat followed by controlled and continuous delivery of a second drug from an osmotic core.

It is yet another object of the present invention to develop a floating osmotic device that maintains high therapeutic activity and better patient compliance.

The dosage form as described in the present invention is effective for immediate release of one drug followed by continuous, controlled delivery of drug present in osmotic core which is capable of acting locally in gastrointestinal tract or acting systemically by absorption via stomach and upper part of the intestine. The rate at which the drug from the osmotic core is released is independent of pH and gastrointestinal motility. The release from the osmotic core depends upon the existence of an osmotic gradient between contents of core and the fluid in the gastrointestinal tract. The drug delivery is essentially constant as long as the osmotic gradient remains constant.

Other features, advantages and objectives of this invention and its preferred embodiments will become apparent from the detailed description and accompanying claims which follows.

### DETAILED DESCRIPTION OF THE INVENTION

The present invention is a novel pharmaceutical composition in the form of a floating osmotic device that is adapted to deliver a first drug from an outer coat upon reaching the gastrointestinal tract, and a second drug from an osmotic core in a controlled manner over a specific time period. The outer coat is also adapted to provide buoyancy for the device, thereby making the device effectively float and remain in the stomach.

The composition of the present invention employs an osmotic system that utilizes the principals of pressure for the controlled delivery of one or more active agents. The release rate of the active agent(s) from the osmotic core is independent of physiological factors of the gastro intestinal tract. The release from the osmotic core depends upon the existence of an osmotic gradient between contents of core and the fluid in the gastrointestinal tract. The drug delivery is essentially constant as long as the osmotic gradient remains constant.

The present invention provides a floating osmotic device comprising a compressed core containing an active agent surrounded by semipermeable membrane, this core is then coated with a mixture of a second active ingredient, a gas generating ingredient, and a gelling polymer.

The present invention preferably provides a floating osmotic device comprising one or more active ingredients, an osmogent, a semipermeable membrane material having a preformed passageway therein, a gas generating ingredient, a swelling agent, and a gelling agent.

A preferred embodiment of the present invention comprises about 3% to about 72% of an active ingredient, 5% to about 10% of an osmogent, about 2% to about 8% of a semipermeable membrane material, about 2.0% to about 10.0% of a plasticizer, about 5% to about 15% of a gas generating material, about 2% to about 10% of a swelling agent, and about 2% to about 10% of a gelling agent. The active ingredient is preferably present in the outer coat in an amount from about 3% to about 12%. The active ingredient is preferably present in the osmotic core in an amount from about 45% to about 60%. As used herein, percentage amounts for an ingredient are the percent weights of the ingredients based on the total weight of the composition, which may be abbreviated as “% w/w.”

The active agent as described in the present invention comprises therapeutic compounds which can be formulated into the present floating osmotic devices include antibacterial substances, anti-inflammatory agents, anti-ulcer agents, antihistamines, antiparasitics, antivirals, proton pump inhibitor, local anesthetics, antifungal, amoebicidal, analgesics, antidepressants, antiarthritics, antiasthmatics, anticoagulants, anticonvulsants, antidiabetics, muscle relaxants, antipsychotics, antihypertensives, antiparkinson agents, hypnotics, sedatives, antispasmodic, tranquilizers, anti-convulsants, muscle contractants, prokinetic agents, anti-microbials, antimalarials, hormonal agents, contraceptives, H2 receptor blockers, diuretics, hypoglycemics, and cardiovascular drugs.

Representative active agents are beta-lactam antibiotics, tetracyclines, chloramphenicol, neomycin, sulfonamides, aminoglycoside antibiotics, nitrofurazone, nalidixic acid, penicillin, tetracycline, glutarimide, oxytetracycline, oxybutanin, chlorotetracycline, erythromycin, cephalosporins, nalidixic acid, ofloxacin, amifloxacin, norfloxacin, ciprofloxacin, pefloxacin, lomefloxacin and salts thereof. Other representative active agents include cisapride, metclopromide, sucralfate, melatonin, carbemezepine, metprolol, propranolol, chloroquine, phenobarbital thiopental, urethanes, spironolactone, furosamide, disulfiram, indepamide, methyl dopa, prazocin, timolol, deserpidine, chorpromazine, fluphenazines, benzodiazepines, benzocaine, lidocaine, tetracaine, diazepam, scopalamine, methocarbamol, mephenesin, procainamide, sodium nitrate, nitroglycerin, atenolol, alprenolol, niacin, folic acid, simvastatin, clonidine hydrochloride, cimitedine, omeprazole, ranitidine, loratidine, 4-aminoquinolines, pyrimethamine, rabiprazole, acyclovir, interferon, betamethasone, prednisolone, captopril, phenylbutazone, metformin, indomethacin, diclofenac, oxyphenbutazone, ibuprofen, chlorpheniramine, orlistat, loratadine, theophylline, pseudoephedrine, warfarin, carvedilol, phenelzine, amitriptyline, tolbutamide, prednisolone, and the salts thereof.

The active ingredient present in the outer coat and the active ingredient present in the osmotic core of the floating osmotic device may be same or different.

When the active agent is of limited solubility in the fluid within the environment of delivery, it is preferable to add osmotically effective solutes, osmotic agents, or osmagents that are capable of being totally or partially solubilized in the fluid within the environment. These osmagents will aid in either the suspension or dissolution of the active agent in the core. Examples of osmagents for use in the present invention include: salts of acids and bases, sugars, sugar alcohols, sodium chloride, potassium chloride, calcium sulfate, sodium sulfite, magnesium chloride, magnesium sulfate, calcium bicarbonate, d-mannitol, sodium sulfate, calcium lactate, urea, sucrose, lactose, dextrose, combinations thereof, and other similar or equivalent materials. An osmagents can also be incorporated to the core of the osmotic device to control the release of an active agent therein. For example, when the agent is only partially or incompletely soluble in the fluid of an environment of delivery, it can be released as a suspension provided sufficient fluid has been imbibed or absorbed by the core to form a suspension. The most preferred osmogent for use in the present invention is sodium chloride present in an amount from about 5.0% to about 10.0%, preferably from about 7.0% to about 9.0%, and most preferably about 8.5% by weight based on the total weight of the composition.

As stated above, the controlled release of the active agent from the osmotic core of the floating osmotic device is independent of pH or gastrointestinal motility, but is dependent upon the existence of an osmotic gradient between the contents of the core and fluid in the gastrointestinal tract. The release of the active agent is essentially constant as long as the osmotic gradient remains constant and then gradually falls to zero. Inert components remain in tablet core and elimination in the feces is the insoluble shell.

The semipermeable membrane is formed of a material that is substantially permeable to the passage of fluid from the environment to the core and is substantially impermeable to the passage of active agent from the core. Examples of semipermeable membrane materials useful in the present invention include cellulose esters, cellulose ethers, and cellulose esters-ethers. Other preferred materials include cellulose diacetate, cellulose triacetate, cellulose acetate, cellulose acetate butyrate, cellulose trimallitate, ethyl cellulose, and methyl methacrylate. A preferred semi-permeable membrane is cellulose triacetate in an amount from about 5.0% to about 10.0%, preferably from about 3.0% to about 7.0%, and most preferably about 5.0% by weight based on the total weight of the composition.

Plasticizers can also be included in the present osmotic device to modify the properties and characteristics of the polymers used in the coats. Plasticizers useful in the invention can be selected from glycol ethers, poly(propylene glycol), block polymers, low molecular weight poly(ethylene glycol), citrate ester-type plasticizers, triacetin, propylene glycol, and glycerin. Such plasticizers can also include ethylene glycol, 1,2-butylene glycol, diethylene glycol, triethylene glycol, and other poly(ethylene glycol) compounds, monopropylene glycol monoisopropyl ether, propylene glycol monoethyl ether, diethylene glycol monoethyl ether, sorbitol lactate, ethyl lactate, butyl lactate, and ethyl glycolate. The most preferred being poly ethylene glycol 400 which is used in an amount preferably from 2.0% to 10% by weight, preferably 3.0% to 8.0% and the most preferred being 5.0% by weight based on the total weight of the composition.

The preformed passageway in the semipermeable wall that communicates the core of the osmotic device with the exterior of the device can be generated by mechanical perforation, laser perforation, or any other suitable method. Although the osmotic device is depicted with a single passageway, a device according to the present invention can comprise at least one or more passageways, as per the need.

The pharmaceutical composition of the present invention comprises gas generating ingredient which generates gas on contact with gastric fluid and is selected from sodium carbonate, sodium bicarbonate, calcium carbonate, and sodium metabisulfte. The most preferred being sodium bicarbonate and is present in an amount from 5% to 15%, preferably from 7.0% to 12.0% and the most preferred being 8.5% by weight based on the total weight of the composition. The gas generating ingredient upon interaction with gastric fluid generates carbon dioxide or sulfur dioxide that gets entrapped within hydrated gel matrix of the gelling agent.

The pharmaceutical composition of the present invention comprises of swelling agent which swells several times greater than its original volume on contact with fluid of the environment. Examples of swelling agents are starch, sodium starch glycolate, crosslinked carboxy methylcellulose, crosslinked polyvinyl pyrrolidone, and partially pregelatinised starch. The most preferred being partially pregelatinised starch which is present in an amount from 2.0% to 10.0%, preferably from 5.0% to 8.0% and the most preferred being 6.0% by weight based on the total weight of the composition.

The pharmaceutical composition of the present invention comprises gelling agent which on contact with fluid of the environment hydrates and forms a viscous gel matrix which traps the gas generated by action of the gas generating ingredient with gastric fluid. Examples of gelling agents are hydroxypropyl methylcellulose, methyl cellulose, hydroxy propylcellulose, carbomer, carboxy methylcellulose, chitosan, and sodium alginate. The most preferred being hydroxypropyl methylcellulose (4000 cps) which is present in an amount from 2.0% to 10.0%, preferably from 3.0% to 7.0% and the most preferred being 4.0% by weight based on the total weight of the composition. In the present invention, the concentration of the gelling agent is adjusted so it does not hinder the release of active ingredient from the outer core, but can form a loose gel matrix that entraps the generated gas.

The osmotic device of the invention can also comprise any other suitable ingredient, such as adsorbents, fillers, antioxidants, buffering agents, colorants, flavorants, sweetening agents, tablet antiadherents, lubricants, tablet binders, diluents, tablet direct compression excipients, tablet disintegrants, tablet glidants, polishing agents, and other equivalent excipients.

According to the present invention, tablets are prepared by mixing the first active ingredient with an osmogent and granulating with polyvinyl pyrrolidone in isopropyl alcohol. The granules are dried, lubricated with magnesium stearate and Aerosil 200, and compressed into tablets, which are coated with cellulose triacetate and polyethylene glycol 400. An orifice is laser drilled through the semi-permeable membrane by a laser drilling machine. The tablets are further compression coated with the mixture of gas generating ingredient, swelling agent, gelling agent, lubricant, and the second active ingredient using a Dry-cota compression coating machine.

### EXAMPLE 1

### Ofloxacin

Example 1 is a tablet according to the present invention wherein the active agent is Ofloxacin, which is required for the treatment of local action on H. pylori in the stomach.

| | | % w/w |
|---|---|---|
| 1. | Ingredients of Osmotic Core | |
| | Ofloxacin | 47.8 |
| | Sodium chloride | 8.5 |
| | Polyvinyl pyrollidone K30 | 3.5 |
| | Magnesium stearate | 0.5 |
| | Aerosil 200 | 0.2 |
| | Cellulose triacetate | 5.0 |
| | Poly Ethylene glycol 400 | 5.0 |
| 2. | Ingredients of Outer Coating | |
| | Ofloxacin | 10.0 |
| | Sodium bicarbonate | 8.5 |
| | Partially pregelatinised starch | 6.0 |
| | Hydroxypropyl methylcellulose 4000 cps | 4.0 |
| | Talc | 1.0 |

The tablet of Example 1 is prepared by mixing Ofloxacin with sodium chloride and granulating with solution of polyvinyl pyrolidone in isopropyl alcohol. The granules are dried, lubricated with magnesium stearate and Aerosil 200, and compressed into tablets, which are coated with cellulose triacetate and polyethylene glycol 400. The passageway is laser drilled. The tablets are further compression coated with the mixture of sodium bicarbonate, partially pregelatinised starch, hydroxypropyl methylcellulose, talc, and Ofloxacin.

The tablet of Example 1 was tested for dissolution in 0.1 N HCl using USP apparatus 1 at 100 rpm. The results are as follows:

| | Ofloxacin released |
|---|---|
| Time (hrs.) | (cumulative %) |
| 1 | 15.5 |
| 2 | 22.0 |
| 4 | 30.3 |
| 6 | 41.15 |
| 8 | 53.45 |
| 10 | 61.19 |
| 12 | 72.31 |
| 14 | 81.97 |
| 16 | 92.78 |
| 18 | 98.78 |

### EXAMPLE 2

Example 2 is a tablet according to the present invention wherein the active agent is Famotidine, which is required for systemic action and absorbed from the upper part of gastrointestinal tract.

| | | % w/w |
|---|---|---|
| 1. | Ingredients of Osmotic Core | |
| | Famotidine | 52.8 |
| | Sodium chloride | 7.0 |
| | Poly vinyl pyrollidone K30 | 2.5 |
| | Magnesium stearate | 0.5 |
| | Aerosil 200 | 0.2 |
| | Cellulose triacetate | 4.5 |
| | Poly Ethylene glycol 400 | 4.0 |
| 2. | Ingredients of Outer Coating | |
| | Famotidine | 7.5 |
| | Sodium bicarbonate | 8.5 |
| | Partially pregelatinised starch | 6.5 |
| | Hydroxypropyl methylcellulose 4000 cps | 5.0 |
| | Talc | 1.0 |

The tablet of Example 2 is prepared by mixing famotidine with sodium chloride and granulating with solution of polyvinyl pyrrolidone in isopropyl alcohol. The granules are dried, lubricated with magnesium stearate and Aerosil 200, and compressed into tablets, which are then coated with cellulose triacetate and polyethylene glycol 400. The passageway is laser drilled. The tablets are further compression coated with the mixture of sodium bicarbonate, partially pregelatinised starch, hydroxypropyl methylcellulose, talc, and famotidine.

The tablet of Example 2 was tested for dissolution in 0.1 N HCl using USP apparatus 1 at 100 rpm. The results are as follows:

| | Famotidine released |
|---|---|
| Time (hrs.) | (cumulative %) |
| 1 | 10.5 |
| 2 | 26.45 |
| 4 | 32.4 |
| 6 | 43.94 |
| 8 | 57.78 |
| 10 | 66.20 |
| 12 | 75.14 |
| 14 | 82.77 |
| 16 | 91.05 |
| 18 | 97.67 |

### EXAMPLE 3

Example 3 is a tablet according to the present invention wherein the active agent is metformin, which is absorbed only from the stomach and the upper part of the gastrointestinal tract.

| | | % w/w |
|---|---|---|
| 1. | Ingredients of Osmotic Core | |
| | Metformin | 48.0 |
| | Sodium chloride | 9.0 |
| | Poly vinyl pyrollidone K30 | 4.0 |
| | Magnesium stearate | 0.5 |
| | Aerosil 200 | 0.2 |
| | Cellulose triacetate | 6.0 |
| | Poly Ethylene glycol 400 | 4.0 |
| 2. | Ingredients of Outer Coating | |
| | Metformin | 9.3 |
| | Sodium bicarbonate | 8.0 |
| | Partially pregelatinised starch | 5.5 |
| | Hydroxypropyl methylcellulose 4000 cps | 4.5 |
| | Talc | 1.0 |

The tablet of Example 3 is prepared by mixing metformin with sodium chloride and granulating with solution of polyvinyl pyrrolidone in isopropyl alcohol. The granules are dried, lubricated with magnesium stearate and Aerosil 200, and compressed into tablets, which are coated with cellulose triacetate and polyethylene glycol 400. The passageway is laser drilled. The tablets are further compression coated with the mixture of sodium bicarbonate, partially pregelatinised starch, hydroxypropyl methylcellulose, talc, and metformin.

The tablet of Example 3 was tested for dissolution in 0.1 N HCl using USP apparatus 1 at 100 rpm. The results are as follows:

| | Metformin released |
|---|---|
| Time (hrs.) | (cumulative %) |
| 1 | 9.0 |
| 2 | 21.23 |
| 4 | 34.42 |
| 6 | 46.12 |
| 8 | 58.12 |
| 10 | 68.33 |
| 12 | 77.42 |
| 14 | 89.87 |
| 16 | 95.41 |

### EXAMPLE 4

Example 4 is a tablet according to the present invention wherein the active agent is domperidone, which is absorbed from the stomach and the upper part of the gastrointestinal tract.

| | | % w/w |
|---|---|---|
| 1. | Ingredients of Osmotic Core | |
| | Domperidone | 56.8 |
| | Sodium chloride | 8.0 |
| | Poly vinyl pyrollidone K30 | 4.0 |
| | Magnesium stearate | 0.5 |
| | Aerosil 200 | 0.2 |
| | Cellulose triacetate | 5.5 |
| | Poly Ethylene glycol 400 | 3.5 |
| 2. | Ingredients of Outer Coating | |
| | Domperidone | 5.0 |
| | Sodium bicarbonate | 7.5 |
| | Partially pregelatinised starch | 5.0 |
| | Hydroxypropyl methylcellulose 4000 cps | 3.0 |
| | Talc | 1.0 |

The tablet of Example 4 is prepared by mixing domperidone with sodium chloride and granulating with solution of polyvinyl pyrolidone in isopropyl alcohol. The granules are dried, lubricated with magnesium stearate and Aerosil 200, and compressed into tablets, which are then coated with cellulose triacetate and polyethylene glycol 400. The passageway is laser drilled. The tablets are further compression coated with the mixture of sodium bicarbonate, partially pregelatinised starch, hydroxypropyl methylcellulose, talc, and domperidone.

The tablet of Example 4 was tested for dissolution in 0.1 N HCl using USP apparatus 1 at 100 rpm. The results are as follows:

| | Domperidone released |
|---|---|
| Time (hrs.) | (cumulative %) |
| 1 | 8.97 |
| 2 | 19.87 |
| 4 | 30.01 |
| 6 | 41.64 |
| 8 | 52.45 |
| 10 | 63.14 |
| 12 | 75.54 |
| 14 | 82.77 |
| 16 | 91.47 |
| 18 | 98.75 |

### EXAMPLE 5

Example 5 is a tablet according to the present invention wherein the active agents are Pseudoephedrine and Cetirizine dihydrochloride. The active ingredient in the outer coat is Cetirizine dihydrochloride, which is released immediately, and the active ingredient in the osmotic core is pseudoephedrine, which is released in a controlled manner.

| | | % w/w |
|---|---|---|
| 1. | Ingredients of Osmotic Core | |
| | Pseudoephedrine | 57.5 |
| | Sodium chloride | 7.0 |
| | Poly vinyl pyrollidone K30 | 3.5 |
| | Magnesium stearate | 0.5 |
| | Aerosil 200 | 0.2 |
| | Cellulose triacetate | 5.0 |
| | Poly Ethylene glycol 400 | 4.0 |
| 2. | Ingredients of Outer Coating | |
| | Cetirizine dihydrochloride | 5.0 |
| | Sodium bicarbonate | 7.0 |
| | Partially pregelatinised starch | 5.3 |
| | Hydroxypropyl methylcellulose 4000 cps | 4.0 |
| | Talc | 1.0 |

The tablet of Example 5 is prepared by mixing pseudoephedrine with sodium chloride and granulating with solution of polyvinyl pyrolidone in isopropyl alcohol. The granules are dried, lubricated with magnesium stearate and Aerosil 200, and compressed into tablets, which are then coated with cellulose triacetate and polyethylene glycol 400. The passageway is laser drilled. The tablets are further compression coated with the mixture of sodium bicarbonate, partially pregelatinised starch, hydroxypropyl methylcellulose, talc, and Cetirizine dihydrochloride.

The tablet of Example 5 was tested for dissolution in 0.1 N HCl using USP apparatus 1 at 100 rpm. The results are as follows:

| | Pseudoephedrine |
|---|---|
| | released |
| Time (hrs.) | (cumulative %) |
| 1 | 8.97 |
| 2 | 19.87 |
| 4 | 30.01 |
| 6 | 41.64 |
| 8 | 52.45 |
| 10 | 63.14 |
| 12 | 75.54 |
| 14 | 82.77 |
| 16 | 91.47 |
| 18 | 98.75 |
| | Cetirizine dihydrochloride |
| | released |
| Time (mins.) | (cumulative %) |
| 5.0 | 21.05 |
| 10.0 | 36.45 |
| 15.0 | 51.78 |
| 20.0 | 68.12 |
| 30.0 | 80.05 |
| 45.0 | 94.85 |

Compositions according to the present invention do not only provide gastric-retentive devices or dosage forms, but also provide the release of active agents in a continuous and controlled manner through an osmotic system. Compositions according to the present invention have an advantage that they may be retained for a long period of time in the stomach of a mammal, thereby delivering a drug over a period of time that is significant for the clinical need. Also compositions according to the present invention have the advantage that they may provide gastric retention in order to improve the absorption of the active agents which are absorbed only from the stomach to jejunum, and also to offer local treatment in the stomach.

It is to be understood that the examples and embodiments described hereinabove are for the purposes of providing a description of the present invention by way of examples and are not to be viewed as limiting the present invention in any way. Modification that may be made to that described in above examples by those of ordinary skill in the art are also contemplated by the present invention and are to be included within the spirit.

What is claimed is:

1. The floating osmotic device comprises of:
a compressed core containing an active agent for controlled delivery;
a semipermeable membrane surrounding the core that is substantially permeable to surrounding fluid and substantially impermeable to the first active agent; and
an outer coating that surrounds the semipermeable membrane and includes a gas generating ingredient, a gelling agent, and an active agent for immediate delivery.

2. The floating osmotic device according to claim 1, wherein the compressed core contains an osmogent.

3. The floating osmotic device according to claim 2, wherein the osmogent is sodium chloride present in an amount from about 5.0% to about 10.0% by weight based on the total weight of the device.

4. The floating device according to claim 1, wherein the compressed core contains an osmogent selected from the group consisting of: salts of acids, salts of bases, sugars, sugar alcohols, sodium chloride, potassium chloride, calcium sulfate, sodium sulfite, magnesium chloride, magnesium sulfate, calcium bicarbonate, d-mannitol, sodium sulfate, calcium lactate, urea, sucrose, lactose, dextrose, and combinations thereof.

5. The floating osmotic device according to claim 1, wherein the semipermeable membrane is selected from the group consisting of: cellulose triacetate, cellulose acetate, cellulose diacetate, cellulose acetate-butyrate, cellulose trimallitate, ethyl cellulose, methyl methacrylate, and combinations thereof.

6. The floating osmotic device according to claim 1, wherein the semipermeable membrane is cellulose triacetate present in an amount from about 2.0% to about 8.0% by weight based on the total weight of the device.

7. The floating osmotic device according to claim 1, wherein the semipermeable membrane includes a plasticizer.

8. The floating osmotic device according to claim 7, wherein the plasticizer is polyethylene glycol 400 present in an amount from about 2.0% to about 10.0% by weight based on the total weight of the composition.

9. The floating osmotic device according to claim 1, wherein the semipermeable membrane includes a plasticizer selected from the group consisting of: glycol ethers, polypropylene glycol, block polymers, low molecular weight polyethylene glycol, citrate ester-type plasticizers, triacetin, propylene glycol, glycerin, ethylene glycol, 1,2-butylene glycol, diethylene glycol, triethylene glycol, monopropylene glycol monoisopropyl ether, propylene glycol monoethyl ether, diethylene glycol monoethyl ether, sorbitol lactate, ethyl lactate, butyl lactate, ethyl glycolate, polyethylene glycol 400, and combinations thereof.

10. The floating osmotic device according to claim 1, wherein the gas generating ingredient is selected from the group consisting of: sodium carbonate, sodium bicarbonate, sodium metabisulfite, calcium carbonate, and combinations thereof.

11. The floating osmotic device according to claim 1, wherein the gas generating ingredient is sodium bicarbonate present in an amount from about 5.0% to about 15.0% by weight based on the total weight of the composition.

12. The floating osmotic device according to claim 1, wherein the outer coating contains a swelling agent.

13. A floating osmotic device according to claim 12, wherein the swelling agent is partially pregelatinised starch present in an amount from about 2% to about 10.0% by weight based on the total weight of the composition.

14. The floating osmotic device according to claim 1, wherein the outer coating contains a swelling agent selected from the group consisting of: starch, sodium starch glycolate, crosslinked carboxy methyl cellulose, crosslinked polyvinyl pyrrolidone, partially pregelatinised starch, and combinations thereof.

15. The floating osmotic device according to claim 1, wherein the gelling agent is selected from the group consisting of: hydroxypropyl methyl cellulose, methyl cellulose, hydroxy propyl cellulose, carbomer, carboxy methyl cellulose, chitosan, sodium alginate, the most preferred being hydroxypropyl methylcellulose.

16. A floating osmotic device according to claim 1, wherein the gelling agent is hydroxypropyl methylcellulose present in an amount from about 3% to about 10.0% by weight based on the total weight of the composition.

17. The floating osmotic device according to claim 1, wherein the gas generating ingredient is adapted to generate a gas in the presence of a fluid, wherein the gel agent is adapted to form a substantially gas-impermeable matrix in the presence of a fluid, and whereby gas generated in the presence of a fluid becomes effectively trapped by the gas-impermeable gel matrix causing the device to float in the fluid.

18. The floating osmotic device according to claim 1, wherein the semipermeable membrane has an orifice that allows a fluid to contact the compressed core, whereby the active ingredient is released continuously and in a controlled manner through the orifice in the presence of a fluid.

19. The floating osmotic device according to claim 1, wherein the floating osmotic device is adapted to be taken into a person's gastric region and upper intestine by being oral administered.

20. The floating osmotic device according to claim 1, wherein the active agent for controlled delivery and the active agent for immediate delivery are both adapted to be absorbed from the stomach or upper part of the small intestine.

21. The floating osmotic device according to claim 1, wherein the active agent for controlled delivery and the active agent for immediate delivery are both useful in treatment of diseases of the stomach.

22. A process for the preparation of a floating osmotic device comprising the steps of:
providing a compressed core containing an active agent for controlled delivery;
surrounding the compressed core with a semipermeable membrane that is substantially permeable to fluid and substantially impermeable to the first active agent;
coating the semipermeable membrane with an outer coating having a gas generating ingredient, a gelling agent, and an active agent for immediate delivery.

23. The process according to claim 22, wherein the compressed core contains an osmogent.

24. The process according to claim 23, wherein the osmogent is sodium chloride present in an amount from about 5.0% to about 10.0% by weight based on the total weight of the device.

25. The process according to claim 22, wherein the compressed core contains an osmogent selected from the group consisting of: salts of acids, salts of bases, sugars, sugar alcohols, sodium chloride, potassium chloride, calcium sulfate, sodium sulfite, magnesium chloride, magnesium sulfate, calcium bicarbonate, d-mannitol, sodium sulfate, calcium lactate, urea, sucrose, lactose, dextrose, and combinations thereof.

26. The process according to claim 22, wherein the semipermeable membrane is selected from the group consisting of: cellulose triacetate, cellulose acetate, cellulose diacetate, cellulose acetate-butyrate, cellulose trimallitate, ethyl cellulose, methyl methacrylate, and combinations thereof.

27. The process according to claim 22, wherein the semipermeable membrane is cellulose triacetate present in an amount from about 2.0% to about 8.0% by weight based on the total weight of the device.

28. The process according to claim 22, wherein the semipermeable membrane includes a plasticizer.

29. The process according to claim 28, wherein the plasticizer is polyethylene glycol 400 present in an amount from about 2.0% to about 10.0% by weight based on the total weight of the composition.

30. The process according to claim 22, wherein the semipermeable membrane includes a plasticizer selected from the group consisting of: glycol ethers, polypropylene glycol, block polymers, low molecular weight polyethylene glycol, citrate ester-type plasticizers, triacetin, propylene glycol, glycerin, ethylene glycol, 1,2-butylene glycol, diethylene glycol, triethylene glycol, monopropylene glycol monoisopropyl ether, propylene glycol monoethyl ether, diethylene glycol monoethyl ether, sorbitol lactate, ethyl lactate, butyl lactate, ethyl glycolate, polyethylene glycol 400, and combinations thereof.

31. The process according to claim 22, wherein the gas generating ingredient is selected from the group consisting of: sodium carbonate, sodium bicarbonate, sodium metabisulfite, calcium carbonate, and combinations thereof.

32. The process according to claim 22, wherein the gas generating ingredient is sodium bicarbonate present in an amount from about 5.0% to about 15.0% by weight based on the total weight of the composition.

33. The process according to claim 22, wherein the outer coating contains a swelling agent.

34. A floating osmotic device according to claim 33, wherein the swelling agent is partially pregelatinised starch present in an amount from about 2% to about 10.0% by weight based on the total weight of the composition.

35. The process according to claim 22, wherein the outer coating contains a swelling agent selected from the group consisting of: starch, sodium starch glycolate, crosslinked carboxy methyl cellulose, crosslinked polyvinyl pyrrolidone, partially pregelatinised starch, and combinations thereof.

36. The process according to claim 22, wherein the gelling agent is selected from the group consisting of: hydroxypropyl methyl cellulose, methyl cellulose, hydroxy propyl cellulose, carbomer, carboxy methyl cellulose, chitosan, sodium alginate, the most preferred being hydroxypropyl methylcellulose.

37. The process according to claim 22, wherein the gelling agent is hydroxypropyl methylcellulose present in an amount from about 3% to about 10.0% by weight based on the total weight of the composition.

38. The process according to claim 22, wherein the gas generating ingredient is adapted to generate a gas in the presence of a fluid, wherein the gel agent is adapted to form a substantially gas-impermeable matrix in the presence of a fluid, and whereby gas generated in the presence of a fluid becomes effectively trapped by the gas-impermeable gel matrix causing the device to float in the fluid.

39. The process according to claim 22, wherein an orifice is created in the semipermeable membrane before the semipermeable membrane is coated with the external coat, the orifice allowing a fluid to contact the compressed core, whereby the active ingredient is released continuously and in a controlled manner through the drilled orifice in the presence of a fluid.

40. The process according to claim 22, wherein the floating osmotic device is adapted to be taken into a person's gastric region and upper intestine by being oral administered.

41. The process according to claim 22, wherein the active agent for controlled delivery and the active agent for immediate delivery are both adapted to be absorbed from the stomach or upper part of the small intestine.

42. The process according to claim 22, wherein the active agent for controlled delivery and the active agent for immediate delivery are both useful in treatment of diseases of the stomach.
